(19) United States
(12) Patent Application Publication
(10) Pub. No.: US 2018/0125860 A1
(43) Pub. Date: May 10, 2018

(54) Contraceptive Compositions and Methods for Improved Efficacy and Modulation of Side Effects

(71) Applicants: Agis KYDONIEUS, Kendall Park, NJ (US); Michael E. KAFRISEN, Palm Beach, FL (US); Katie MACFARLANE, Washington, DC (US); Agile Therapeutics, Inc., Princeton, NJ (US)

(72) Inventors: Agis Kydonieus, Kendall Park, NJ (US); Michael E. Kafrissen, Palm Beach, FL (US); Katie Macfarlane, Washington, DC (US)

(73) Assignee: Agile Therapeutics, Inc., Princeton, NJ (US)

(21) Appl. No.: 15/574,698

(22) PCT Filed: May 18, 2016
(86) PCT No.: PCT/US2016/033024

§ 371 Date: Nov. 16, 2017

Related U.S. Application Data
(60) Provisional application No. 62/163,113, filed on May 18, 2015.
(60) Provisional application No. 62/254,999, filed on Nov. 13, 2015.

(51) Int. Cl. A61K 31/57; A61K 31/565; A61P 15/18

(52) U.S. Cl. CPC A61K 31/57; A61K 2300/00; A61P 15/18; A61K 31/565

(57) ABSTRACT

Compositions and methods for the delivery of progestin hormones that have binding affinity to the Sex Hormone Binding Globulin (SHBG) are disclosed. The compositions combine such progestins with non-progestin SHBG ligands to displace at least part of the progestin from SHBG in the blood plasma, thereby increasing its bioavailability. Also disclosed are methods to modulate progestin and estrogen levels in the blood through the use of SHBG binding and displacement, to optimize the effectiveness of formulations for contraception and minimize the side effects and adverse events.

### FIELD OF THE INVENTION

This invention is in the field of transdermal delivery of hormones. More specifically, it pertains to the delivery of progestin hormones that have binding affinity to the Sex Hormone Binding Globulin (SHBG) and most specifically to the circulation in blood plasma of modulated levels of unbound progestin hormones, especially of the progestin levonorgestrel (LNG). It also pertains to the modulation of progestin and estrogen levels to optimize the effectiveness of formulations for contraception and minimize the side effects and adverse events.

### BACKGROUND OF THE INVENTION

1. Transdermal Delivery

Transdermal drug delivery systems offer significant advantages over more conventional oral or parenteral dosage forms. First, the administration of the drug is non-invasive and does not require a procedure by a healthcare professional when compared to implants, intrauterine devices (IUD) and injections. Second, the delivery can be for one week from a single patch as compared with oral products that have to be taken every day. Third, transdermal delivery of the drug bypasses the hepatic first pass which metabolizes and inactivates many drugs, including hormones. Fourth, the delivery of the drug is controlled without peaks and valleys, resulting in better side effect profiles, effectiveness and compliance.

Hormone products for contraception include both progestins and estrogens. Many different progestins are used in contraceptive products, but ethinyl estradiol (EE) is almost exclusively used as the estrogenic component of the formulation. Only one transdermal patch is available commercially in the United States, Xulane (the generic equivalent to the Evra patch, which is no longer commercially available), for the delivery of hormones for contraception. This patch delivers the progestin norelgestromin and the synthetic estrogen, EE. It is an efficacious product but it delivers very high amounts of ethinyl estradiol (about 50-60 picograms per milliliter (pg/ml) mean serum concentration), which has been shown to increase the risk of venous thromboembolytic events as well increased side effects, such as breast tenderness and nausea. Delivery of lower levels of EE in contraceptive products (20-30 pg/ml) has been accepted by the U.S. Food and Drug Administration (FDA) as well as the industry as being a good way to formulate for reduction of side effects and better safety profile.

The most widely used progestin is levonorgestrel (LNG) due to its very large safety and efficacy database. There are no commercial transdermal patches delivering only progestins without an estrogenic component such as EE.

2. Estrogen and Progestin Hormones

Estrogens are steroidal estrogen receptor agonists that, under natural conditions, are responsible for development and regulation of the female reproductive system and secondary sex characteristics. For purposes this invention, estrogens include synthetic derivatives of naturally occurring estrogens.

Estrogenic activity is shared by many steroidal and nonsteroidal compounds. The most potent naturally-occurring steroids are 17-beta-estradiol (estradiol) followed by estrone and estriol. Some synthetic steroidal estrogens include EE, mestranol and quinestol. The chemical alterations of the natural estrogens render them effective orally. For example the oral bioavailability of the natural hormone 17-beta estradiol and several of its ester prodrugs is less than 10% (Lokind, K. B et al., (1991) Int. J. Pharmaceutics, 76, 177-182), while the oral bioavailability of the synthetic hormone EE is 95%. The phenolic feature of these compounds is one chemical part of the structure that provides high selective affinity for the estrogen receptors. Nonsteroidal compounds with estrogenic activity occur naturally in plants. These include flavone, isoflavone and coumestan derivatives and they are phenolic compounds mimicking the phenolic ring of the steroids (Goodman and Gilman's The Pharmacological Basis of Therapeutics, 8th edition, Eds Alfred Goodman Gilman, Theodore W. Rail, Alan S. Nies, and Palmer Taylor. P. 1384, New York, Pergamon Press, 1990, p. 1384).

Contraceptive estrogens are used mainly for the regulation of the menstrual cycle. They also aid the control of fertility by preferentially binding to SHBG and displacing the bound progestin, thus allowing higher amounts of free progestin hormones circulating in the plasma.

The natural progestin is progesterone, which has low oral bioavailability. Chemical modifications have produced a variety of orally effective progestins, including hydroxyl-progesterone, medroxyprogesterone, ethynodiol diacetate, norethindrone, nerethynodrel megestrol and LNG, among many others. The main function of progestin hormones is to control fertility.

Though most are orally bioavailable, progestin hormones in general are well known to have poor skin permeation potential, which is an issue with transdermal delivery (see for example US Pub. No. 2013/0317462). There are several patents issued and pending pertaining to the skin permeation of different progestin hormones, both unenhanced as well as using chemical enhancers to increase the skin permeation, typically through the use of a cadaver skin assay. For example, U.S. Pat. No. 5,474,783 discloses the flux of Norethindrone Acetate as being only 0.05 micrograms/cm²/hr; US Pub No. 2007/0098775A1 shows the unenhanced flux of Norelgestromin as being between 0.02 and 0.05 micrograms/cm²/hr and the enhanced flux between 0.3 and 0.9 micrograms/cm²/hr. US Pub. No. 2013/0317462 discloses the unenhanced flux of norethisterone acetate as being 0.05 micrograms/cm²/hr and the enhanced flux as being between 0.1 and 0.13; also the unenhanced flux of nesterone as being 0.005 micrograms/cm²/hr and the enhanced flux as being 0.01 micrograms/cm²/hr. Several other patents describe the permeation through human skin of other progestins: WO 1996/040355A1 presents the unenhanced flux of 17 deacetylnorgestimate as being 0.1 micrograms/cm²/hr and the enhanced flux as being between 0.2 and 0.8 micrograms/cm²/hr; U.S. Pat. No. 4,863,738 discloses the unenhanced flux of progesterone as being 0.14 micrograms/cm²/hr and the unenhanced flux of LNG as being between 0.13 and 0.21 micrograms/cm²/hr. In comparison, U.S. Pat. Nos. 7,045,145 and 7,384,650 disclose an enhanced skin (enhancement with four chemical enhancers) permeation of between 0.25 and 0.3 micrograms/cm²/hr for LNG. It is evident from the above mentioned flux numbers that the permeation of most progestins is very low; therefore it can be important to be able to increase the free progestin levels circulating in the blood plasma.

It is also well known that, in transdermal delivery, the drug being delivered reaches its highest plasma concentration several hours after application of the patch and in the case of hormones this lag time is between one and two days after application of the patch (LNG patch U.S. Pat. No. 7,045,145 B1; Xulane™ norelgestromin/ethinyl estradiol transdermal system prescribing information, Clinical Pharmacology). The standard contraceptive regimen comprises three weeks of hormone treatment and one week of drug free interval with the cycle being repeated every 28 days.

### SUMMARY OF THE INVENTION

There is an optimal amount of progestin that will make a dosage effective for contraception. For oral dosing this level of progestin can be easily delivered because progestins have rapid absorption through the intestinal mucosal tissue. In transdermal delivery, this becomes a major obstacle because the permeation of most progestins through skin is very limited and the patches would need to be large in size and thus difficult to adhere to skin and cosmetically not acceptable.

Advantageously, the inventors have found that the amount of free progestin circulating in the plasma of women can be increased by increasing the amount of estrogen co-delivered, without increasing the amount of progestin delivered. As exemplified herein for the progestin LNG (See table 1 and FIG. 1), the additional delivery of 1 microgram of LNG increases the amount of LNG circulating in the blood plasma by about 3.5 pg/ml, but the additional delivery of 1 microgram of EE, at constant delivery of LNG, increases the amount of LNG circulating in the blood plasma about 30 pg/ml. Since estrogens such as EE, 17-beta estradiol and other hormonal and non-hormonal estrogenic compounds can often be delivered through human skin in substantially higher amounts than LNG and most other progestins, contraception can be achieved in some cases through the use of smaller transdermal patches.

In certain illustrative embodiments of the invention, more than one estrogenic hormone is delivered in the transdermal formulation so as to modulate the appropriate contraceptive efficacy, but also minimize the side effects and adverse events attributed to hormones. Although the experimental work described herein was performed with EE being the estrogenic hormone, the invention is useful with other compounds such as a) natural and synthetic estrogens, b) other hormonal and non-hormonal chemical ingredients with binding affinity to SHBG, c) fragments and small molecules with binding affinity to SHBG and d) hormonal and non-hormonal ingredients that decrease the amount of free SHBG circulating in the blood plasma, thus decreasing the amount of progestin that can bind to SHBG, allowing for larger amounts of free progestin. The above mentioned ingredients and combinations thereof are defined herein as “SHBG ligands”. An important aspect of this use of SHBG ligands is that it allows for the combination of hormonal and non-hormonal compounds, so as to increase the free progestin levels without increasing the side effects that may be caused by pure hormonal compounds. In addition, suitable progestins for use in this invention are those that have some binding affinity to SHBG. For example, since EE has also high affinity to SHBG, it will displace the bound progestin from SHBG and thus increase the free progestin circulating in the plasma. As seen from the examples below, the inventors have found experimentally that for every 10 micrograms per day of EE delivered, the amount of free LNG circulating in the plasma is increased by 300 picograms per ml without increasing the amount of levonorgestrel delivered.

Thus, one aspect of the invention features a contraceptive composition for internal administration to a woman who is at risk of becoming pregnant comprising (a) a progestin with binding affinity to sex hormone binding globulin (SHBG) and (b) one or more non-progestin SHBG ligands that bind to SHBG in an amount sufficient to displace at least a portion of the progestin bound to SHBG, thereby increasing the amount of unbound progestin circulating in the blood plasma of the woman, wherein if the non-progestin SHBG ligand is an estrogen, then the composition is formulated to deliver less than 10 micrograms of the estrogen per day.

In various embodiments, the progestin is norgestrel, levonorgestrel, norethindrone, norethindrone acetate or norethrynodrel.

The non-progestin SHBG ligand, or one of multiple SHBG ligands, can be ethinyl estradiol (EE) and the composition may be formulated to deliver less than 2.5 micrograms of the estrogen per day. In certain embodiments, the composition comprises at least one non-progestin SHBG ligand other than EE, wherein the amount of the SHBG ligand included is an amount equivalent to the amount of EE required to achieve the same portion of displacement of the progestin from the SHBG. Alternatively, the composition can comprise two or more non-progestin SHBG ligands other than EE, wherein the sum of the amounts of the SHBG ligands included is an amount equivalent to the amount of EE required to achieve the same portion of displacement of the progestin from the SHBG.

In certain embodiments, the SHBG ligand is not an estrogen and is an estrogenic compound, a non-estrogenic hormone, an anti-SHBG antibody or fragment thereof, a small molecule, or a combination thereof. As used herein, the term “antibody fragment” refers generally to a polypeptide comprising the CDR of an anti-SHBG antibody, e.g., Fab and a scFv, such that the fragment binds to SHBG.

The SHBG ligand can be combination of an estrogen with one or more of an estrogenic compound, a non-estrogenic hormone, an anti-SHBG antibody or fragment or a small molecule. In particular, the estrogen can include EE or 17 beta estradiol in combination with other SHBG ligands. More particularly, the estrogen includes 17-beta estradiol in combination with estrone and/or estriol. Alternatively, the SHBG ligand is EE or 17-beta estradiol, in combination with a non-estrogen SHBG ligand.

The above-described compositions can be formulated for administration by a route selected from oral, transmucosal, transdermal and subcutaneous. In certain embodiments, the composition is formulated in a transdermal delivery device comprising an active ingredient (AI) layer containing the progestin and the non-progestin SHBG ligand, wherein the AI layer has a skin-contacting surface and a non-skin-contacting surface, and the device further comprises a backing layer adjacent the non-skin-contacting surface. In particular embodiments, the AI layer of the device has a skin-contacting surface of 15 cm² or less, or of 10 cm² or less. In other embodiments, the composition is formulated for oral administration as a tablet or capsule.

Another aspect of the invention features a method of contraception, comprising, during a treatment cycle having a pre-determined treatment interval in which contraceptively effective amounts of progestin hormone are delivered, and a pre-determined rest interval in which no hormone or low dose hormones are delivered, administering to a woman a during the treatment interval a contraceptive composition comprising (a) a progestin with binding affinity to sex hormone binding globulin (SHBG) and (b) one or more non-progestin SHBG ligands that bind to SHBG in an amount sufficient to displace at least a portion of the progestin bound to SHBG, thereby increasing the amount of unbound progestin circulating in the blood plasma of the woman, wherein if the non-progestin SHBG ligand is an estrogen, then the composition is formulated to deliver less than 10 micrograms of the estrogen per day. The treatment cycle typically is composed of a treatment interval of between three and twelve weeks, followed by a one-week rest interval.

In various embodiments of the method, the progestin is norgestrel, levonorgestrel, norethindrone, norethindrone acetate or norethrynodrel.

The non-progestin SHBG ligand, or one of multiple SHBG ligands, can be ethinyl estradiol (EE) and the composition may be formulated to deliver less than 2.5 micrograms of the estrogen per day. In certain embodiments, the composition comprises at least one non-progestin SHBG ligand other than EE, wherein the amount of the SHBG ligand included is an amount equivalent to the amount of EE required to achieve the same portion of displacement of the progestin from the SHBG. Alternatively, the composition can comprise two or more non-progestin SHBG ligands other than EE, wherein the sum of the amounts of the SHBG ligands included is an amount equivalent to the amount of EE required to achieve the same portion of displacement of the progestin from the SHBG.

In certain embodiments, the SHBG ligand is not an estrogen and is an estrogenic compound, a non-estrogenic hormone, an anti-SHBG antibody or fragment thereof, a small molecule, or a combination thereof.

The SHBG ligand can be combination of an estrogen with one or more of an estrogenic compound, a non-estrogenic hormone, an anti-SHBG antibody or fragment or a small molecule. In particular, the estrogen can include EE or 17 beta estradiol in combination with other SHBG ligands. More particularly, the estrogen includes 17-beta estradiol in combination with estrone and/or estriol. Alternatively, the SHBG ligand is EE or 17-beta estradiol, in combination with a non-estrogen SHBG ligand.

In the above-described methods, the compositions can be formulated for administration by a route selected from oral, transmucosal, transdermal and subcutaneous. In certain embodiments, the composition is formulated in a transdermal delivery device comprising an active ingredient (AI) layer containing the progestin and the non-progestin SHBG ligand, wherein the AI layer has a skin-contacting surface and a non-skin-contacting surface, and the device further comprises a backing layer adjacent the non-skin-contacting surface. In particular embodiments, the AI layer of the device has a skin-contacting surface of 15 cm² or less, or of 10 cm² or less. In other embodiments, the composition is formulated for oral administration as a tablet or capsule.

Another aspect of the invention features a kit for practicing a contraceptive method comprising a treatment cycle having a pre-determined treatment interval in which contraceptively effective amounts of progestin hormone are delivered, and a pre-determined rest interval in which no hormone or low dose hormones are delivered. The kit typically comprises: (a) a multiplicity of treatment interval dosage units sufficient for one or more treatment intervals, wherein the treatment interval dosage units comprise (ii) a progestin with binding affinity to sex hormone binding globulin (SHBG) and (ii) one or more non-progestin SHBG ligands that bind to SHBG in an amount sufficient to displace at least a portion of the progestin bound to SHBG, thereby increasing the amount of unbound progestin circulating in the blood plasma of the woman, wherein if the SHBG ligand is an estrogen, the composition is formulated to deliver less than 10 micrograms of estrogen per day; (b) one or more rest interval dosage units sufficient for the rest interval, wherein the rest interval dosage units comprise (i) no hormone, or (ii) low dose hormone; and (c) instructions for practicing a contraceptive method comprising a treatment cycle having a pre-determined treatment interval in which contraceptively effective amounts of progestin hormone are delivered, and a pre-determined rest interval in which no hormone or low dose hormones are delivered.

In certain embodiments, the kit comprises dosage units for a treatment cycle comprising a treatment interval of between three and twelve weeks, followed by a one-week rest interval. For instance, the kit can contain 21 or a multiple of 21 oral treatment interval dosage units for daily administration and 7 or a multiple of 7 oral rest interval dosage units comprising no hormone or low hormone. Alternatively, the kit may contain 3 or a multiple of 3 transdermal treatment interval dosage units for successive weekly application and 1 or a multiple of 1 rest interval dosage unit comprising low hormone or no hormone. The same multiple for rest interval dosage units may be included, or the multiple may be different, depending on the length of treatment interval.

In various embodiments of the kit, the progestin is norgestrel, levonorgestrel, norethindrone, norethindrone acetate or norethrynodrel.

The non-progestin SHBG ligand, or one of multiple SHBG ligands, can be ethinyl estradiol (EE) and the composition may be formulated to deliver less than 2.5 micrograms of the estrogen per day. In certain embodiments, the composition comprises at least one non-progestin SHBG ligand other than EE, wherein the amount of the SHBG ligand included is an amount equivalent to the amount of EE required to achieve the same portion of displacement of the progestin from the SHBG. Alternatively, the composition can comprise two or more non-progestin SHBG ligands other than EE, wherein the sum of the amounts of the SHBG ligands included is an amount equivalent to the amount of EE required to achieve the same portion of displacement of the progestin from the SHBG.

In certain embodiments, the SHBG ligand is not an estrogen and is an estrogenic compound, a non-estrogenic hormone, an anti-SHBG antibody or fragment thereof, a small molecule, or a combination thereof.

The SHBG ligand can be combination of an estrogen with one or more of an estrogenic compound, a non-estrogenic hormone, an anti-SHBG antibody or fragment or a small molecule. In particular, the estrogen can include EE or 17 beta estradiol in combination with other SHBG ligands. More particularly, the estrogen includes 17-beta estradiol in combination with estrone and/or estriol. Alternatively, the SHBG ligand is EE or 17-beta estradiol, in combination with a non-estrogen SHBG ligand.

Another aspect of the invention features method of contraception that is sometimes referred to as “on demand” contraception. The method comprises: (a) administering to a woman on a regular or continuous basis, during a treatment cycle of duration selected by the woman, a progestin with binding affinity to sex hormone binding globulin (SHBG); and (b) in a time proximity of between about 12 hours before and about 6 hours after the woman engages in sexual intercourse, administering to the woman a bolus of one or more non-progestin SHBG ligands that bind to SHBG in an amount sufficient to displace at least a portion of the progestin bound to SHBG, thereby increasing the amount of unbound progestin circulating in the blood plasma of the woman, thereby increasing the contraceptive efficacy of the progestin being administered on the regular or continuous basis during the time frame in which the woman could become pregnant due to engaging in sexual intercourse.

In certain embodiments of this method, the treatment cycle comprises a treatment interval of between three and twelve weeks, followed by a one-week rest interval in which no hormone is administered, or in which low dose hormone is administered.

The progestin can be selected from norgestrel, levonorgestrel, norethindrone, norethindrone acetate, or norethrynodrel in various embodiments.

In certain embodiments, the bolus of the SHBG ligand delivered to the woman comprises the equivalent of about 20-100 micrograms of EE. The bolus

In this method, the progestin can be formulated in a composition for administration by a route selected from oral, transmucosal, transdermal and subcutaneous. In certain embodiments, the progestin is formulated in a transdermal delivery device comprising an active ingredient (AI) layer containing the progestin, wherein the AI layer has a skin-contacting surface and a non-skin-contacting surface, and the device further comprises a backing layer adjacent the non-skin-contacting surface. The progestin also can be formulated in a composition that further comprises a non-progestin SHBG ligand in an amount that delivers an equivalent of less than 10 micrograms per day of EE.

In embodiments of the method, the bolus of SHBG ligand is formulated for oral delivery.

Another aspect of the invention features a kit for practicing an “on demand” contraceptive regimen. The kit comprises: (a) a multiplicity of dosage units of progestin having SHBG binding affinity formulated in a composition for regular or continuous administration via oral, transmucosal, subcutaneous or transdermal delivery; (b) a multiplicity of dosage units of non-progestin SHBG ligand formulated in a composition for administration as a bolus via oral delivery; and (c) instructions for use of the kit components in method of contraception comprising: (i) administering to a woman on a regular or continuous basis, during a treatment cycle of duration selected by the woman, a progestin with binding affinity to sex hormone binding globulin (SHBG); and (ii) in a time proximity of between about 12 hours before and about 6 hours after the woman engages in sexual intercourse, administering to the woman a bolus of one or more non-progestin SHBG ligands that bind to SHBG in an amount sufficient to displace at least a portion of the progestin bound to SHBG, thereby increasing the amount of unbound progestin circulating in the blood plasma of the woman, thereby increasing the contraceptive efficacy of the progestin being administered on the regular or continuous basis during the time frame in which the woman could become pregnant due to engaging in sexual intercourse.

The kit may utilize a progestin selected from norgestrel, levonorgestrel, norethindrone, norethindrone acetate, or norethrynodrel in various embodiments. In certain embodiments, the progestin is formulated in a composition that further comprises another SHBG ligand in an amount that delivers an equivalent of less than 10 micrograms per day of EE.

In certain embodiments, the bolus of the SHBG ligand delivered to the woman comprises the equivalent of about 20-100 micrograms of EE.

In certain embodiments, the progestin composition is formulated in a transdermal delivery device comprising an active ingredient (AI) layer containing the progestin, wherein the AI layer has a skin-contacting surface and a non-skin-contacting surface, and the device further comprises a backing layer adjacent the non-skin-contacting surface.

Another aspect of the invention features a method of increasing the amount of circulating progestin in the serum of a patient administered a progestin, comprising: (a) administering to the patient a progestin having binding affinity to sex hormone binding globulin (SHBG), whereby upon delivery of the progestin to the serum of the patient, at least a portion of the progestin is bound to the SHBG and thereby sequestered from circulation in the patient's serum; and (b) co-administering to the patient one or more non-progestin SHBG ligands in an amount sufficient to displace at least part of the progestin from SHBG in the patient's serum, thereby increasing the amount of circulating progestin in the serum of the patient.

Yet another aspect of the invention provides a method of increasing the potency of a progestin that binds to SHBG, said method comprising co-administering the progestin with a subclinical amount of a non-progestin SHBG ligand other than a progestin.

Still another aspect of the invention features a method of increasing the contraceptive efficacy of a progestin that binds to SHBG, said method comprising co-administering the progestin with a subclinical amount of a non-progestin SHBG ligand.

In any one of the above-described three methods, the non-progestin SHBG ligand can be an estrogen and can be administered in an amount that results in delivery of less than 10 micrograms per day of the estrogen. In various embodiments, the estrogen is EE and is administered in an amount that results in delivery of less than 2.5 micrograms per day of the EE.

Other features and advantages of the invention will be understood from the drawings, description and examples set forth herein.

### BRIEF DESCRIPTION OF THE DRAWINGS

FIG. 1 is a graph showing the effect of EE delivery on LNG plasma levels. Hormones were delivered from a transdermal delivery system as described in the Examples. X axis represents the amount of EE delivered (micrograms per day); Y axis represents the mean serum concentration of LNG (picograms per milliliter), with the 1420 value extrapolated from the data collected.

FIG. 2 is a graph showing in vitro permeation of LNG through human cadaver skin, in the presence of EE (closed circles) or in the absence of EE (open squares). X axis represents time (hours); Y axis represents LNG permeation (micrograms per cm²).

### DETAILED DESCRIPTION OF THE INVENTION

Most progestins also bind to SHBG and thus much of the hormone delivered to the blood is not freely available to provide the contraceptive effect needed. Thus, the present invention pertains to progestins that have binding affinity to SHBG, but which can be displaced by the use of EE, 17-beta estradiol or other SHBG ligands. By “displace” it is meant that the SHBG ligand will occupy binding sites on SHBG that could otherwise bind to the progestin. Such displacement can reduce the amount of progestin bound to SHBG in the plasma, thereby effectively increasing the exposure to the progestin. For example such progestins in order of binding affinity to SHBG include d-norgestrel, dl-norgestrel, norethisterone, LNG, norethynodrel and lynestrenol (including salts, e.g., norethisterone acetate). All of these progestins have higher affinity for SHBG than EE. Megestrol acetate and medroxyprogesterone do not have high binding affinity. (Victor, A., et al., J. Clin. Endocrinol. Metab. 43: 244, 1975). Other publications have shown similar results, e.g., Phillips (1990, Steroids, 55(8):373-375) showed that norgestimate and its metabolites have low binding affinity for SHBG but gestodene, levonorgestrel and 3-keto desogestrel have reasonably high affinities. Schoultz (1989, Gynecol. Obstet. Invest. 27: 151-154) has shown that LNG and norethisterone have high binding affinity to SHBG, but medroxyprogesterone acetate and desogestrel have lesser binding affinity. Pollow (1989, Contraception, 40(3): 325-341) has shown that gestodene, LNG and 3-keto desogestrel have high binding affinity to SHBG, but progesterone, medroxyprogesterone acetate, cyproterone acetate and desogestrel have lesser binding affinity.

SHBG is a glycoprotein that binds to androgens and estrogens. For example, testosterone and estradiol circulate in the blood stream, bound mostly to SHBG. Only a very small fraction of about 1 to 2% is unbound, or free, and thus biologically active and able to activate a cell's receptors. The relative binding affinity of various sex hormones for SHBG has been reported to be dihydrotestosterone>testosterone>androstenediol>estradiol>estrone (Somboonporn, W. & S. Davis, 2004, Endocrine Reviews 25: 374-88). As mentioned in the above paragraph commercially important progestins such as LNG and gestodene also bind to SHBG. The inventors surprisingly realized that the incorporation in transdermal contraceptive formulations of higher amounts of EE, 17-beta estradiol, other SHBG ligands or combinations thereof, will in general produce higher amounts of free progestin circulating in the blood plasma. This hypothesis was proven to be correct as can be seen from the examples shown below. The increase in unbound progestin can allow one to prepare contraceptively effective transdermal patches that are smaller in size and cosmetically more appealing. Further, whether delivered transdermally, orally, or otherwise, the usage of the right SHBG ligand or combination of SHBG ligands could improve contraceptive effectiveness and at the same time modulate side effects and adverse events.

The present invention is particularly useful for progestins that have high binding affinity to SHBG. However, it will also be effective with progestins that have lower binding affinity to SHBG, though at least some binding affinity is needed in order for the SHBG ligand to affect the amount of progestin circulating in the plasma versus bound to SHBG.

Although in the examples shown below the displacing agent (inhibiting binding of the progestin to SHBG) that was used was EE, other agents that can displace the progestin from SHBG are also useful with the invention. Therefore, it should be understood that SHBG ligands other than EE can be used in the same manner, as can other agents, natural or synthetic, even non-steroidal agents (see, e.g., Pugeat, M M et al., 1989, J. Clin. Endocrinol. Metab. 53: 69-75) that bind to SHBG can be used. Such other agents might include, e.g., anti-SHBG antibody or fragments thereof (including polypeptides comprising the relevant complementarity determining regions of such antibodies, e.g., Fab, scFv, and other polypeptides), natural non-steroidal compounds with SHBG binding activity such as flavones, flavanones, isoflavone, isoflavanones, chalcones, parabens diphenylethylene derivatives, bibenzyl derivatives, stilbene derivatives, various mycoestrogens, coumestan derivatives and small molecules (see, e.g., Cherkasov, A. et al., 2005, J. Med. Chem. 48: 3203-3213; Cherkasov et al., 2005(b), J. Chem. Inf. Model 45: 1842-1853; Cherkasov et al., 2006, J. Med. Chem 49: 7466-7478; Cherkasov et al., 2008, J. Med. Chem 51: 2047-2056; Avvakumov et al., 2010, Mol. Cell. Endocrinol. 316: 13-23; Herzog, A G et al., 1991, Epilepsia, 32(4):550-553; Victor, A et al., 1977, Br. Med. J. 8 October, 934-935; Goodman and Gilman, Eight Ed., supra, p. 1384; Hong, H et al., 2015, Toxicol. Sci. 143: 333-348), with phenol being a useful structural indicator of SHBG binding (Hong et al., 2015, supra).

Cherkasov et al. (2005 et seq., supra) describe “in silico” drug design methodologies for screening large numbers of compounds. The authors report having discovered 29 nonsteroidal SHBG ligands having affinity for SHBG with IC₅₀ concentrations from about 13 micromolar to about 125 micromolar using such methods. Hong et al. (2015) report screening 125 structurally diverse chemicals and identifying 87 of these as having affinity for SHBG with IC₅₀ concentrations ranging from about 0.2 nanomolar to about 4 millimolar. While any of these are potentially useful in embodiments of this invention, SHBG ligands with affinities at the higher end of this range (e.g., IC₅₀ of 100 micromolar or less, or 50 micromolar or less, or 10 micromolar or less, or even 1 micromolar or less) may offer advantages. Additionally, it may be desirable to select SHBG ligands capable of displacing more or less than 50% of a progestin bound to SHBG; for instance 10%, 20%, 30%, 40%, 60%, 70%, 80% or 90%.

The skilled artisan can easily test the binding affinity of a proposed SHBG ligand by methods well known in the art, and typically utilize competitive binding assays using testosterone or estradiol as the reference compound (see, e.g., Chersakov et al., 2005, supra; Hong et al., 2015, supra). For example, Cherkasov et al. (2005, supra), used an established competitive ligand binding assay to determine the binding affinities of test compounds to human SHBG in relation to those of testosterone and estradiol. The assay involved mixing diluted human pregnancy serum containing SHBG with tritium-labeled dihydrotestosterone (DHT) as the labeled ligand, then testing each compound for its ability to displace the labeled DHT from the SHBG. The IC₅₀ concentrations for the test compounds could be determined from the resulting competition curves. Concentrations that would achieve more or less than 50% displacement of progestin from SHBG may also be selected on the basis of concentration curve information, such as that provided in the literature mentioned above.

As alluded to above, the binding affinities of hormones (testosterone, progestins, estrogens) for SHBG have been found to vary. In utilizing both hormonal and non-hormonal SHBG ligands in the present invention, it is most useful to begin with the particular progestin being employed and then determine the amount of a selected SHBG ligand needed based on a reference substance, such as EE. For instance, the inventors have demonstrated for LNG that an increase of 10 micrograms per day of EE delivered increases LNG in the blood by 300 picograms per milliliter. Accordingly, if it is desired to use a different SHBG ligand to achieve the same result, the amount of that ligand can be made equivalent to the amount of EE, calculated by their respective binding affinities for SHBG. In this regard, it is noteworthy that the binding affinity of 17 β-estradiol for SHBG is upward of 60-fold greater than that of EE, therefore proportionately less 17 β-estradiol would be needed to achieve the same effect as seen for EE. Conversely, the binding affinities of a number of non-hormonal SHBG ligands has been shown to be tenfold or more lower than that of EE. The comparative binding affinities in testosterone displacement assays, can be used to determine how much of a selected SHBG ligand is needed to achieve the same result as a selected amount of EE (see, e.g., Schotter, M & G. Spitzeller, 1998 J. Nat. Prod. 61: 119-121; Nilsson, B & B. von Schoultz, 1989, Gynecol. Obstet Invest. 27:151-154; Phillips, A et al., 1990, Steroids 55:373-375; also Cherkasov et al., 2005, 2005(b), 2006 and 2008, supra). As mentioned earlier, Cherkasov et al. and other groups have measured the SHBG binding affinities of thousands of diverse compounds and have thereby identified dozens to hundreds of compounds suitable for use in the compositions and methods of the present invention.

Thus, for LNG, an amount of SHBG ligand other than EE can be selected based on relative affinity compared to EE (or other reference). For instance, if the binding affinity of a selected SHBG ligand is 10-fold less than that of EE, then this would require co-delivery of 10 micrograms of the selected SHBG ligand in place of each microgram of EE. So, if raising the free progestin concentration by 30 pg/ml requires addition of one microgram of EE, the same effect can be obtained by administering 10 micrograms of a different SHBG ligand that has 10-fold less affinity for SHBG. The multiplication product of the binding affinity of a specific ligand by the amount or concentration of the ligand should yield the same result for all ligands, based on the selected progestin. If a different progestin is selected, the same exercise will result in selecting appropriate amounts of that progestin, comparing the SHBG binding affinity of that progestin with the binding affinity of LNG.

When EE, 17 β-estradiol or another estrogen typically used for human contraceptive or other hormone therapy is selected as the SHBG ligand, the present invention has the distinct advantage of enabling use of substantially less hormone than typically used for contraception. In preferred embodiments the compositions of the present invention are formulated to deliver less than 10 micrograms per day of these estrogens. In particular embodiments, they are formulated to deliver less than 9.5 micrograms per day, or less than 9 micrograms per day, or less than 8.5 micrograms per day, or less than 8 micrograms per day, or less than 7.5 micrograms per day, or less than 7 micrograms per day, or less than 6.5 micrograms per day, or less than 6 micrograms per day, or less than 5.5 micrograms per day, or less than 5 micrograms per day, or less than 4.5 micrograms per day, or less than 4 micrograms per day, or less than 3.5 micrograms per day, or less than 3 micrograms per day, or less than 2.5 micrograms per day, or less than 2 micrograms per day, or less than 1.5 micrograms per day, or less than 1 microgram per day of EE. In other embodiments, for instance using 17 β-estradiol, even less estrogen can be delivered, for instance less than 2 micrograms per day, or less than 1.5 microgram per day, or less than 1 microgram per day, or less than 900 nanograms per day, or less than 800 nanograms per day, or less than 700 nanograms, or less than 600 nanograms per day, or less than 500 nanograms per day, or less than 400 nanograms per day, or less than 300 nanograms per day, or less than 200 nanograms per day, or less than 100 nanograms per day. Of course, similar amounts can be calculated for other naturally occurring or synthetic estrogens, based on their binding affinity for SHBG.

The compositions of the present invention can be formulated for administration via a variety of routes known to the person of skill in the art, including oral, transmucosal (e.g., sublingual, thin film) and transdermal. The compositions can also be formulated within long-acting reversible contraceptive (LARC) devices, such as intrauterine devices (IUDs) and implants. Oral and sublingual dosage may be particularly suitable for delivery of SHBG ligands having a lesser binding affinity for SHBG than, for instance EE or 17 β-estradiol, since larger amounts of such ligands may be needed that cannot be effectively delivered by other routes.

Pharmaceutical formulations or preparations containing the compositions of the invention and a suitable carrier can be solid dosage forms which includes tablets, capsules, cachets, pellets, pills, powders or granules; topical dosage forms which include solutions, powders, fluid emulsions, fluid suspensions, semi-solids, ointments, pastes, creams, gels or jellies, foams and controlled release depot entities; transdermals, vaginal rings, buccal formulations; and implants.

It is known in the art that active ingredients are formulated into compositions with pharmaceutically acceptable diluents, fillers, disintegrants, binders, lubricants, surfactants, hydrophobic vehicles, water soluble vehicles, emulsifiers, buffers, humectants, moisturizers, solubilizers, antioxidants preservatives and the like. Numerous pharmacologic references are available for guidance, e.g., “Modern Pharmaceutics”, Banker & Rhodes, Marcel Dekker, Inc. (1979); “Goodman & Gilman's The Pharmaceutical Basis of Therapeutics”, 8th Edition, MacMillan Publishing Co., New York (1980), or Remington's Pharmaceutical Sciences, Osol, A., ed., Mack Publishing Company, Easton, Pa. (1980).

As mentioned, transdermal compositions may represent an interesting embodiment of the present invention because the invention enables the use of lesser amounts of hormones to result in delivery of an effective amount of hormone. Thus, as mentioned throughout this specification, transdermal delivery of adequate amounts of progestin may be achieved through the use of a smaller size transdermal delivery vehicle. In various embodiments, the skin-contacting surface area of the hormone-delivering part of a transdermal patch may be 20 cm² or less. In certain embodiments, the surface area may be 19 cm² or less, or 18 cm² or less, or 17 cm² or less, or 16 cm² or less, or 15 cm² or less, or 14 cm² or less, or 13 cm² or less, or 12.5 cm² or less, or 12 cm² or less, or 11 cm² or less, or 10 cm² or less, or 9 cm² or less, or 8 cm² or less, or 7 cm² or less, or 6 cm² or less, or 5 cm² or less. The invention in some embodiments may also be used to decrease the size of other delivery vehicles, e.g., implants, tablets, and the like.

Transdermal compositions are formulated in accordance with well known methods, depending on the selected hormones to be delivered. In an exemplary embodiment, LNG is delivered from a transdermal delivery system comprising an adhesive polymer matrix and one or more skin permeation enhancers and other excipients as described in the Examples (see also U.S. Pat. Nos. 7,045,145 and 7,384,650). Delivery of other progestins can also be accomplished, with or without the use of skin permeation enhancers (see, e.g., WO 2013/112806 A2).

The compositions of the invention are preferably produced in the form of a kit or package, with the daily (e.g., for oral) or weekly (e.g., for transdermal) dosages arranged for proper sequential administration. Thus, other illustrative embodiments of the invention provide, a pharmaceutical package that contains the contraceptive compositions in multiple dosage units in a synchronized, fixed sequence, wherein the sequence or arrangement of the dosage units corresponds to the stages of daily or weekly administration. In certain embodiments, such kits or packages contain placebos for use during a withdrawal interval between contraceptive treatments. These are referred to herein as “rest intervals” between “treatment intervals,” collectively comprising a “treatment cycle.” The placebos can take any form, including a different size or color of dosage form (e.g., pill or patch) that contains no contraceptively effective amounts of components. Alternatively, the package can contain “blanks,” such as, for instance, seven out of 28 blisters in a blister pack of oral dosage forms, or one out of four compartments in a transdermal package, being empty.

In other illustrative embodiments, the invention can improve the currently available “progestin-only” hormone delivery option for contraception, i.e., by using a second SHBG ligand to reduce binding of progestin to SHBG in the blood, thereby increasing its availability. For many women, a progestin-only patch may be a better treatment option when compared to the combined oral contraceptives. Accordingly, a product of the present invention would be more appropriate when estrogen-containing contraceptives are contraindicated or otherwise inappropriate or unwanted. The target population includes women who are breast feeding or hypertensive, are at increased risk of thrombosis, experience vascular migraine headaches; overweight women with body mass index (BMI) >30 kg/m²; or women who are cigarette smokers over 35 years of age.

Similarly, LARC devices, such as IUDs and implants, are typically formulated to contain only progestin. These devices can be supplemented with a non-progestin SHBG ligand to increase the amount of circulating progestin delivered from the devices and increase their efficacy.

The inventors performed a clinical trial toward the development of a progestin-only patch. The patches in addition to the hormone LNG contained four enhancers, a humectant polyvinyl pyrrolidone/vinyl acetate copolymer and an acrylate pressure sensitive adhesive comprising about 60% of the patch active layer. The patches were heat sealed in a polyester film with a Barex interior surface. The patches were saturated with the drug LNG and each patch delivered LNG for seven days. Two size patches were produced at 6.5 and 12.5 cm², delivering 43 and 83 mg per day of LNG; otherwise the patches were identical. It was observed that the average plasma levels of LNG were low at 174 and 307 picograms per ml with average Cₘᵢₙ values of 109 and 205 picograms per ml. It is accepted that for LNG-only contraceptive products, average LNG levels of 250 picograms per ml plasma should be reached to obtain effective products (Contraception 1997 November 56 (5): 317-321). An active patch portion of over 20 cm² will have to be used to reach the minimum blood levels required for all patients. Since a peripheral adhesive will have to be used to obtain seven day adhesion, the actual patch will be even larger, e.g., almost 30 cm², which is aesthetically unacceptable to many women. Thus, our invention can provide effective transdermal hormone contraceptive patches of small size, which comprise mainly progestins with an SHBG ligand such as ethinyl estradiol or a non-hormonal SHBG ligand. If the affinity of the SHBG ligand is approximately equivalent to or greater than that of EE (IC₅₀=0.8 micromolar, according to Hong (2015)), then very small amounts of the SHBG ligand can be employed. For EE, these amounts are in the range of less than 10 micrograms per day or less, e.g., less than 1, 2, 3, 4, 5, 6, 7, 8, or 9 micrograms per day, as stated above. In the case of EE or other estrogen SHBG ligand, such a formulation, despite delivering only very low amounts of the estrogen SHBG ligand, may offer further advantages in better controlling the cycle parameters such as breakthrough bleeding and spotting and withdrawal flow, since estrogens generally control these cycle parameters.

In another illustrative embodiment, the invention can extend the progestin-only patch contraceptive approach described above to an “on demand” contraception, controlled by the female herself The approach would be to use a progestin only patch that may not be required to deliver as much progestin as patches intended for extended wear, e.g., a full week (so as to be smaller and cosmetically more acceptable to the female). Prior to sexual intercourse, preferably a period of 0 to 12 hours prior to intercourse the female takes a bolus dosage containing between 10 and 50 micrograms of EE or another SHBG ligand of equivalent strength. The progestin levels are going to increase several fold above the basal levels (Contraception, 1992 March: 45(3):187). In this way the female has control of the amount of estrogen that she takes, which can have some significant side effects as described above. This method of contraception can be used to advantage by women who have sexual intercourse intermittently, such as 4 to 6 times per month or less, for instance. In this way the high and long exposure to estrogenic components is minimized, as compared to those of the standard hormone contraceptives. SHBG ligands in the isoflavone class are suitable ligands for this aspect of the invention, though the estrogens described above and any other SHBG ligand can be used.

In this aspect of the invention, it may be advantageous to package the continuous or regularly-administered progestin together with the bolus dose of SHBG ligand together in a kit. For the progestin, the dosage form may be oral or transmucosal, but it is preferred to be transdermal. For the SHBG ligand, the dosage may be in the form of a pill, thin film, sublingual dosage or other form that allows for delivery of the bolus without much delay. Thus, for example, a kit may contain multiple transdermal progestin patches and multiples of SHBG ligand pills. In the regimen, the woman wears the progestin patch continuously, replacing one for another in accordance with package directions. When the woman anticipates engaging in sexual intercourse, she ingests one (or the directed number) of bolus dosages of SHBG ligand, while continuing to wear the progestin patch. The woman may take a break from the regimen at her discretion by removing the progestin patch, thereby initiating a rest interval in which a withdrawal bleed may be experienced.

In a second clinical trial, two LNG plus EE transdermal patches were prepared and tested. These patches were identical in thickness, as well as content of LNG, enhancers, humectants and pressure sensitive adhesive per square centimeter of patch. The only difference between these patches and the LNG-only patches mentioned above was the addition in these patches of 1.8 and 2.3 mg of EE respectively. Surprisingly, the clinical data from these patches showed that the addition of EE increased the LNG plasma levels about ten times more than the addition of an equivalent amount of LNG. Thus the co-delivery of 5 micrograms of EE per day in the LNG 6.5 patch will increase the LNG levels above the minimum required level of 250 picograms LNG per ml. Thus a patch about three times smaller than a LNG-only patch can be obtained with the desired properties, by co-delivering with LNG an ultralow amount of about 1 to 10 micrograms of EE per day. Other estrogens or non-hormonal SHBG ligands could also be used. For example the natural estrogen 17-beta estradiol could be used with the delivery of very small amounts of less than 10 micrograms per day. As will be shown below, extremely smaller amounts of 17-beta estradiol could be used, as its potency is 60-fold higher than that of EE in displacing progestins from SHBG. Also, combination of SHBG ligands can be delivered to optimize an effective patch size and also reduce the side effects and adverse events. Such low amount can be a subclinical amount, i.e., an amount that does not cause observable clinical effects; e.g., if the SHBG ligand is ethinyl estradiol, then a subclinical amount is an amount that does not noticeably affect the woman's menstrual cycle or cause side effects such as breast tenderness and nausea commonly associated with ethinyl estradiol.

In some illustrative embodiments, the invention uses the natural hormone 17 β-estradiol (estradiol) in formulations of the invention, alone or in combination with other SHBG ligands, because as it was mentioned above it is at least 60-fold more potent than EE, the estrogen used almost exclusively in contraception (Hong et al., 2015, supra, measured EE and 17 b-estradiol in a testosterone displacement assay, and found 17 b-estradiol to be more than 100-fold more potent than EE, i.e., IC50 of 7×10⁻⁹M versus 7.9×10⁻⁷M). The relative affinity of estradiol versus ethinyl estradiol has been studied and shown that estradiol is 60-fold more potent than ethinyl estradiol in replacing testosterone from SHBG, and it has 60-fold higher affinity than ethinyl estradiol to testosterone binding globulin (J, Clin. Endocrinol. Metab. 43: 244, 1976; J Clin. Endocrin. Metab. 53 no 1, 69, 1981). Thus, by this comparison, the EE equivalent of 10 micrograms per day is 10/60=0.17 mg (167 nanograms) per day in the case of estradiol. Therefore, substantially smaller transdermal patches can be prepared using estradiol versus those that are commonly prepared using ethinyl estradiol. Estradiol has not been used in oral contraception due to its high first pass hepatic metabolism which in humans is about 95%. In transdermal delivery, since there is no hepatic first pass effect, 30 micrograms delivered will correspond to approximately 30 micrograms circulating systemically.

In some illustrative embodiments, the invention uses 17-beta estradiol, alone or in combination with other SHBG ligands to increase the amount of free progestin circulating in the plasma and thus allow for the preparation of effective but small size transdermal patches. Small size patches are a major advantage for the acceptance of transdermal contraceptive patches by women. Estradiol has high binding affinity to the SHBG and can effectively displace the progestin hormones that are also bound to SHBG.

In some illustrative embodiments, the invention uses a combination of natural estrogenic SHBG ligands together with estradiol to allow for the tailoring of the available free progestin, depending on the ratio of estradiol to the other natural SHBG ligand used. For example the most potent naturally occurring estrogen in humans is estradiol, followed by estrone, estriol (Goodman and Gilman, 8th ed. p. 1384) and estetrol. As mentioned above other natural non-estrogenic compounds such as flavone and isoflavone can be used in combination with estradiol.

As discussed above a typical transdermal dosing regimen useful in the practice of this invention comprises successive application of 3 one-week patches each comprising a contraceptively effective amount of a progestin and a SHBG ligand or combination of 2 or more SHBG ligands, followed by a one week rest interval during which no hormones or SHBG ligands are delivered. However, the invention can be practiced with any other dosing regimen including, e.g., extended cycle dosing regimens or modified rest interval regimens.

As mentioned above, the inventors performed several clinical trials toward the development of LNG-only or LNG plus EE containing transdermal contraceptive patches. They have clearly shown that the estrogenic component of the contraceptive formulation is not only important for regulation of the menstrual cycle, but it is equally as important for the control of fertility. These examples are shown below and clearly describe embodiments of my invention.

To make certain that the substantial increase of free LNG levels that were observed with the small increase in EE levels was not due to pharmacokinetic differences between the LNG-only patches and the LNG plus EE patches, an in vitro skin permeation experiment was run using human cadaver skin. The purpose of this experiment was to determine if the amount of LNG that permeated through human skin was different between the LNG-only patches and the LNG plus EE patches. The results are shown in example 3 and indicate that the permeation of LNG from both patches was the same. The presence of EE in one of the patches did not affect the permeation of LNG through human skin and thus the LNG pharmacokinetics between the two patches. This further substantiated the fact that our invention was due to pharmacodynamic effects of the differential binding of the EE and LNG hormones to SHBG.

While the benefits of the present invention are greatest in the case of transdermal administration, the invention can also be applied to vaginal administration of contraception, oral contraceptives, wherein a woman is administered a contraceptively effective amount of a progestin during a treatment interval and is also administered an amount of one or more SHBG ligands during the treatment interval. Combined transdermal and oral delivery is also contemplated, e.g., transdermal delivery of 17-beta estradiol only, with oral delivery of the progestin LNG.

Illustrative embodiments of the invention include a transdermal polymeric matrix comprising a pressure sensitive adhesive, a progestin, and a non-progestin SHBG ligand, having one or more of the following features. In certain embodiments, the SHBG ligand is (i) a compound that does not bind to estrogen receptors or binds only poorly to estrogen receptors such that the binding to estrogen receptors is clinically irrelevant; or (ii) estradiol, estrone, estriol, estetrolestradiol, or ethinyl estradiol and the amount of the SHBG ligand delivered into the subject's plasma is a very low amount, i.e., an amount that is contraceptively ineffective; or (iii) estradiol, estrone, estriol, estetrolestradiol, or ethinyl estradiol and the amount of the SHBG ligand delivered into the subject's plasma is 1 to 10 micrograms per day EE equivalent.

In certain embodiments, which may or may not be combined with those set forth above, the progestin is LNG. In particular embodiments, the composition is formulated to deliver at least about 20-30 micrograms per day LNG, or at between 40-70 micrograms per day, or from 80 to 120 micrograms per day, for seven or more days. In certain embodiments, some of which are applicable to transdermal delivery of LNG, the polymeric matrix further comprises one or more permeation enhancers.

In certain embodiments, the polymeric matrix is comprised within a transdermal patch having a surface area <20 cm2. In certain embodiments, the matrix is comprised within a transdermal patch and adheres to the skin for seven days.

Specific illustrative embodiments include, e.g.: (i) a transdermal polymeric matrix comprising a pressure sensitive adhesive, a progestin, and 0.1 to 1 mg EE; or (ii) a transdermal polymeric matrix comprising a pressure sensitive adhesive, 2 to 2.5 mg LNG, and 0.1 to 1 mg EE, wherein the composition delivers 30 or more micrograms per day of LNG.

Other illustrative embodiments of the invention include a tablet or capsule, a progestin, and a non-progestin SHBG ligand, having one or more of the following features. In certain embodiments, the SHBG ligand is (i) a compound that does not bind to estrogen receptors or binds only poorly to estrogen receptors such that the binding to estrogen receptors is clinically irrelevant (i.e., the amount is subclinical); or (ii) estradiol, estrone, estriol, estetrolestradiol, or ethinyl estradiol and the amount of the SHBG ligand delivered into the subject's plasma is a very low amount, i.e., an amount that is contraceptively ineffective (i.e., the amount is subclinical); or (iii) estradiol, estrone, estriol, estetrolestradiol, or ethinyl estradiol and the amount of the SHBG ligand delivered into the subject's plasma is 1 to 10 micrograms per day EE equivalent.

In certain embodiments, which may or may not be combined with those set forth above, the progestin is LNG. In other embodiments, the progestin is norgestrel, norethindrone, norethindrone acetate, or norethrynodrel.

Specific illustrative embodiments include tablets or capsules containing, e.g.: (i) 0.1 to 0.15 milligrams LNG, or (ii) 0.3 to 0.5 milligrams norgestrel, or (iii) 0.4 to 1.5 milligrams norethindrone; each combined with (i) 0.5 to 10 micrograms of EE, or (ii) 0.01 to 0.5 micrograms of estradiol.

The following examples describe the invention in greater detail. They are intended to illustrate, rather than to limit, the invention.

### Example 1

In this clinical trial LNG only patches were prepared and used. The patches in addition to the hormone LNG contained four enhancers, a humectant polyvinyl pyrrolidone/vinyl acetate copolymer and an acrylate pressure sensitive adhesive comprising about 60% of the patch active layer. The patches were heat sealed in an aluminum/polyester film with a Barex (acrylonitrile/methyl acrylate copolymer) interior surface. The patches were saturated with the drug LNG and each patch delivered LNG for seven days. Two size patches were produced at 6.5 and 12.5 cm², delivering 43 and 83 micrograms per day of LNG; otherwise the patches were identical. This was a randomized, open-label, parallel group study. Thirty-six (36) subjects were enrolled and 35 completed the study (17 in the 6.5 low dose group and 18 in the 12.5 high dose group). Each patch was administered as a continuous regimen, with weekly patch applications and no patch-free intervals. Each patch was applied to the abdomen. The patch was changed weekly for the entire study duration (up to 8 weeks).

The average plasma levels of LNG were low at 174 and 307 picograms per ml respectively. Another interesting point is the fact that the additional 40 micrograms per day of LNG (going from LNG6.5 to LNG12.5) did not increase the LNG blood levels very much, corresponding to only about 3 picograms per ml of plasma for every microgram of LNG delivered. Since the patches are saturated with LNG, a very large size patch will have to be developed to provide for an effective LNG-only contraceptive product.

### Example 2

In a second clinical trial, two LNG plus EE transdermal patches were prepared and tested. These patches were identical in thickness, as well as content of LNG, enhancers, humectants and pressure sensitive adhesive per square centimeter of patch. The only difference between these patches and the LNG-only patches mentioned above was the addition in these patches of 1.8 and 2.3 mgs of EE per patch respectively. The objective of this study was to evaluate the pharmacodynamic effects on ovulation suppression and cycle control of patches containing different doses of ethinyl estradiol during three cycles of administration, as well as obtain serum concentrations of LNG and EE during the study. Enrollment included 45 subjects in each of the LNG/EE12.5L and the LNG/EE12.5H treatment groups, which are the pertinent groups to this invention.

The results showed that the average LNG blood plasma levels were 786 and 1012 picograms per ml respectively and the average blood plasma levels of EE were 15.4 and 23.4 picograms per ml respectively.

Pertinent pharmacokinetic results from the clinical data obtained from Examples 1 and 2 shown above are presented in Table 1.

TABLE 1
Clinical Data Results, Pharmacokinetic, Contraceptive
and Cycle Control Parameters

| | LNG6.5 | LNG12.5 | LNG/EE12.5L | LNG/EE12.5H |
|---|---|---|---|---|
| LNG Delivered | 43 | 83 | 83 | 83 |
| (μg) | | | | |
| EE Delivered (μg) | 0 | 0 | 16.7 | 25 |
| Average LNG | 174 | 307 | 786 | 1012 |
| Blood levels | | | | |
| (pg/ml) | | | | |
| Average EE | 0 | 0 | 15.4 | 23.4 |
| Blood levels | | | | |
| (pg/ml) | | | | |

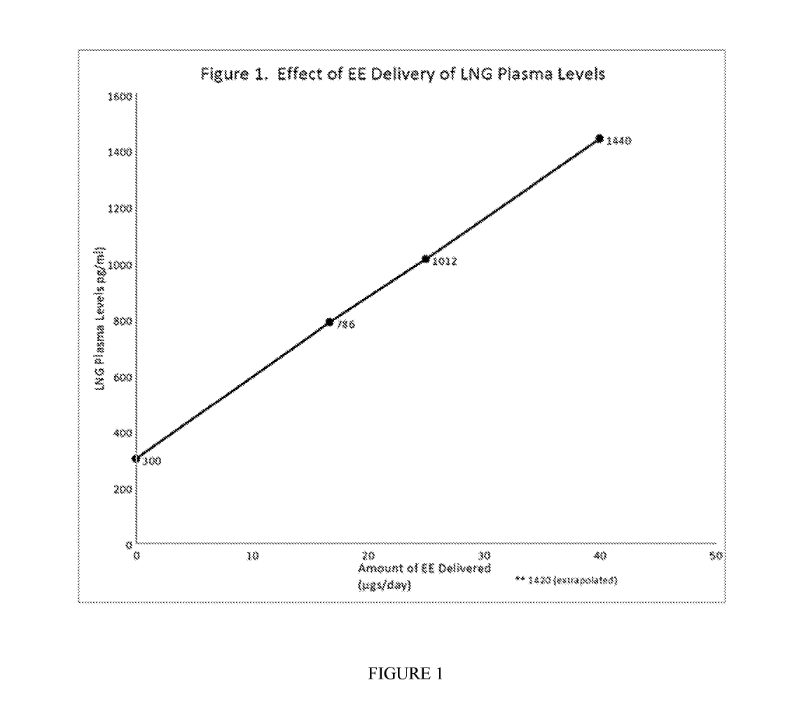

The amount of LNG in blood plasma as a function of micrograms of EE delivered is shown in FIG. 1. This figure shows that at constant LNG delivery from the patch the amount of LNG in the blood plasma is substantially higher when co-delivering EE. While keeping the LNG delivery from the patch constant, for every 10 micrograms of EE co-delivered the amount of LNG in the blood plasma increases by about 300 picograms per ml.

### Example 3

The inventors hypothesized that the high release of LNG into the blood stream when EE was co-administered was due to the fact that EE has high affinity to SHBG and thus it displaces the LNG that was bound to SHBG. To eliminate the possibility that the delivery of LNG from the LNG-only patches is different from that from patches containing both LNG and EE, an in-vitro skin permeation experiment was performed using patches from columns 2 and 4 shown in Table 1.

The objective of this study was to compare in vitro skin permeation of LNG through human cadaver skin from a patch containing both EE and LNG (Patch of column 4, Table 1) with a patch containing only LNG (patch in column 2, Table 1).

Only one (1) skin donor was used in these in vitro skin permeation experiments (split thickness dermatomed approximately at 375 μm human cadaver skin). All in vitro skin permeation studies were conducted using the PermeGear Membrane Transport System. Each Membrane Transport System consisted of vertical, jacketed (37° C.±0.5° C.) Franz diffusion cells with magnetic stirrer, 2 chamber (donor and receiver) sets of 6 units, orifice diameter 15 mm, effective permeation surface area 1.767 cm². Each cell was prepared by cutting the skin samples into approximately 3 cm×3 cm squares and mounting them on the top of the receiver compartment of Franz cells. Patches to be tested were placed on the stratum corneum surface of the skin and the donor and receiver compartments of the diffusion cells were then clamped in place. The receiver compartment was then filled with approximately 12 mL of receiver medium/receptor solution (Phosphate Buffered Saline (PBS)+0.5% Oleth 20+0.008% Gentamicin, pH 7.3), tilting to make sure the receiver medium and the skin/solution interface was free of any air bubbles.

Skin flux studies were run for a period of 168 hours. At predetermined intervals (24, 48, 72, 96, 120, 144, and 168 hours) after starting the experiment, the entire contents of the receiver compartment were collected for determination of LNG concentration using HPLC. The LNG solubility in the receiver medium was sufficient to ensure sink conditions throughout each collection interval.

The results of these experiments are shown in Table 2 for the LNG plus EE containing patches and in Table 3 for the patches containing only LNG.

TABLE 2
Cumulative Amount of Levonorgestrel Permeated
as a Function of Time (LNG plus EE patch)

| | Amount Permeated/Unit Area | | | | | | | |
|---|---|---|---|---|---|---|---|---|
| | (μg/cm²) | | | | | | | Steady |
| | Time (hours) | | | | | | | State Flux |
| | 24 | 48 | 72 | 96 | 120 | 144 | 168 | (μg/cm²/hr) |
| | 5.4 | 12.5 | 19.3 | 26.4 | 32.7 | 38.9 | 45.0 | 0.275 |
| Mean (SD) | 0.78 | 1.19 | 1.57 | 2.07 | 2.50 | 2.90 | 3.32 | 0.019 |

TABLE 3
Cumulative Amount of Levonorgestrel Permeated
as a Function of Time (LNG-only patch)

| | Amount Permeated/Unit Area | | | | | | | |
|---|---|---|---|---|---|---|---|---|
| | (μg/cm²) | | | | | | | Steady |
| | Time (hours) | | | | | | | State Flux |
| | 24 | 48 | 72 | 96 | 120 | 144 | 168 | (μg/cm²/hr) |
| | 7.1 | 15.3 | 23.1 | 30.9 | 37.9 | 44.5 | 50.8 | 0.304 |
| Mean (SD) | 1.22 | 2.27 | 3.27 | 4.17 | 4.89 | 5.57 | 6.17 | 0.036 |

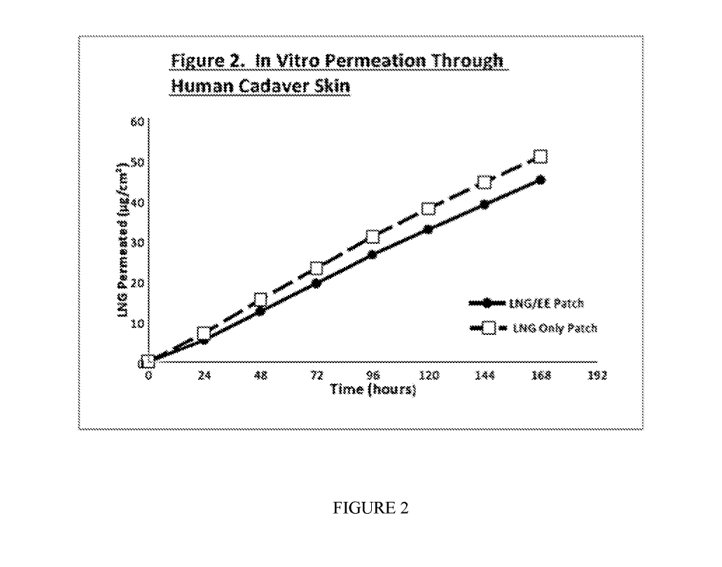

It is obvious by looking at tables 2 and 3 that the release of LNG is not affected by the presence of EE. This is also shown graphically in FIG. 2.

The present invention is not limited to the embodiments described and exemplified herein. It is capable of variation and modification within the scope of the appended claims. The term “includes,” “including,” or the like is meant to be non-limiting. All technical articles, scientific papers, patents, patent publications and the like cited herein are incorporated by reference in their entireties.

1. A contraceptive composition for internal administration to a woman who is at risk of becoming pregnant comprising (a) a progestin with binding affinity to sex hormone binding globulin (SHBG) and (b) one or more non-progestin SHBG ligands that bind to SHBG in an amount sufficient to displace at least a portion of the progestin bound to SHBG, thereby increasing the amount of unbound progestin circulating in the blood plasma of the woman, wherein if the non-progestin SHBG ligand is an estrogen, then the composition is formulated to deliver less than 10 micrograms of the estrogen per day.

2. The composition of claim 1, wherein the non-progestin SHBG ligand, or one of multiple SHBG ligands, is ethinyl estradiol (EE) and wherein the composition is formulated to deliver less than 2.5 micrograms of the estrogen per day.

3. The composition of claim 1, comprising at least one non-progestin SHBG ligand other than EE, wherein the amount of the SHBG ligand included is an amount equivalent to the amount of EE required to achieve the same portion of displacement of the progestin from the SHBG.

4. The composition of claim 1, comprising two or more non-progestin SHBG ligands other than EE, wherein the sum of the amounts of the SHBG ligands included is an amount equivalent to the amount of EE required to achieve the same portion of displacement of the progestin from the SHBG.

5. The composition of claim 1, wherein the progestin is norgestrel, levonorgestrel, norethindrone, norethindrone acetate or norethrynodrel.

6. The composition of claim 1, wherein the SHBG ligand is not an estrogen and is an estrogenic compound, a non-estrogenic hormone, an anti-SHBG antibody or fragment thereof, a small molecule, or a combination thereof.

7. The composition of claim 1, wherein the SHBG ligand is a combination of an estrogen with one or more of an estrogenic compound, a non-estrogenic hormone, an anti-SHBG antibody or fragment or a small molecule.

8. The composition of claim 7, wherein the estrogen includes EE or 17 beta estradiol in combination with other SHBG ligands.

9. The composition of claim 8, wherein the estrogen includes 17-beta estradiol in combination with estrone and/or estriol.

10. The composition of claim 1, wherein the SHBG ligand is EE or 17-beta estradiol, in combination with a non-estrogen SHBG ligand.

11. The composition of claim 1, formulated for administration by a route selected from oral, transmucosal, transdermal and subcutaneous.

12. The composition of claim 11, formulated in a transdermal delivery device comprising an active ingredient (AI) layer containing the progestin and the non-progestin SHBG ligand, wherein the AI layer has a skin-contacting surface and a non-skin-contacting surface, and the device further comprises a backing layer adjacent the non-skin-contacting surface.

13. The composition of claim 12, wherein the AI layer of the device has a skin-contacting surface of 15 cm² or less.

14. The composition of claim 10, wherein the AI layer of the device has a skin-contacting surface of 10 cm² or less.

15. The composition of claim 11, formulated for oral administration as a tablet or capsule.

16. A method of contraception, comprising, during a treatment cycle having a pre-determined treatment interval in which contraceptively effective amounts of progestin hormone are delivered, and a pre-determined rest interval in which no hormone or low dose hormones are delivered, administering to a woman a during the treatment interval a contraceptive composition comprising (a) a progestin with binding affinity to sex hormone binding globulin (SHBG) and (b) one or more non-progestin SHBG ligands that bind to SHBG in an amount sufficient to displace at least a portion of the progestin bound to SHBG, thereby increasing the amount of unbound progestin circulating in the blood plasma of the woman, wherein if the non-progestin SHBG ligand is an estrogen, then the composition is formulated to deliver less than 10 micrograms of the estrogen per day.

17. The method of claim 16, wherein the treatment cycle comprises a treatment interval of between three and twelve weeks, followed by a one-week rest interval.

18. The method of claim 16, wherein the non-progestin SHBG ligand, or one of multiple SHBG ligands, is ethinyl estradiol (EE) and wherein the composition is formulated to deliver less than 2.5 micrograms of the estrogen per day.

19. The method of claim 16, wherein the composition comprises at least one non-progestin SHBG ligand other than EE, wherein the amount of the SHBG ligand included is an amount equivalent to the amount of EE required to achieve the same portion of displacement of the progestin from the SHBG.

20. The method of claim 16, wherein the composition comprises two or more non-progestin SHBG ligands other than EE, wherein the sum of the amounts of the SHBG ligands included is an amount equivalent to the amount of EE required to achieve the same portion of displacement of the progestin from the SHBG.

21. The method of claim 16, wherein the progestin is norgestrel, levonorgestrel, norethindrone, norethindrone acetate, or norethrynodrel.

22. The method of claim 16, wherein the SHBG ligand is not an estrogen and is an estrogenic compound, a non-estrogenic hormone, an anti-SHBG antibody or fragment thereof, a small molecule, or a combination thereof.

23. The method of claim 16, wherein the SHBG ligand is a combination of an estrogen with one or more of an estrogenic compound, a non-estrogenic hormone, an anti-SHBG antibody or fragment or a small molecule.

24. The method of claim 23, wherein the estrogen includes EE or 17 beta estradiol in combination with other SHBG ligands.

25. The method of claim 24, wherein the estrogen includes 17-beta estradiol in combination with estrone and/or estriol.

26. The method of claim 16, wherein the SHBG ligand is EE or 17-beta estradiol, in combination with a non-estrogen SHBG ligand.

27. The method of claim 16, wherein the composition is formulated for administration by a route selected from oral, transmucosal, transdermal and subcutaneous.

28. The method of claim 27, wherein the composition is formulated in a transdermal delivery device comprising an active ingredient (AI) layer containing the progestin and the non-progestin SHBG ligand, wherein the AI layer has a skin-contacting surface and a non-skin-contacting surface, and the device further comprises a backing layer adjacent the non-skin-contacting surface.

29. The method of claim 28, wherein the AI layer of the device has a skin-contacting surface of 15 cm² or less.

30. The method of claim 28, wherein the AI layer of the device has a skin-contacting surface of 10 cm² or less.

31. The method of claim 27, wherein the composition is formulated for oral administration as a tablet or capsule.

32. A kit for practicing a contraceptive method comprising a treatment cycle having a pre-determined treatment interval in which contraceptively effective amounts of progestin hormone are delivered, and a pre-determined rest interval in which no hormone or low dose hormones are delivered, the kit comprising:
(a) a multiplicity of treatment interval dosage units sufficient for one or more treatment intervals, wherein the treatment interval dosage units comprise (ii) a progestin with binding affinity to sex hormone binding globulin (SHBG) and (ii) one or more non-progestin SHBG ligands that bind to SHBG in an amount sufficient to displace at least a portion of the progestin bound to SHBG, thereby increasing the amount of unbound progestin circulating in the blood plasma of the woman, wherein if the SHBG ligand is an estrogen, the composition is formulated to deliver less than 10 micrograms of estrogen per day;
(b) one or more rest interval dosage units sufficient for the rest interval, wherein the rest interval dosage units comprise (i) no hormone, or (ii) low dose hormone; and
(c) instructions for practicing a contraceptive method comprising a treatment cycle having a pre-determined treatment interval in which contraceptively effective amounts of progestin hormone are delivered, and a pre-determined rest interval in which no hormone or low dose hormones are delivered.

33. The kit of claim 32, comprising dosage units for a treatment cycle comprising a treatment interval of between three and twelve weeks, followed by a one-week rest interval.

34. The kit of claim 33, comprising 21 or a multiple of 21 oral treatment interval dosage units for daily administration and 7 or a multiple of 7 oral rest interval dosage units comprising no hormone or low hormone.

35. The kit of claim 33, comprising 3 or a multiple of 3 transdermal treatment interval dosage units for successive weekly application and 1 or a multiple of 1 rest interval dosage unit comprising low hormone or no hormone.

36. The kit of claim 32, wherein the non-progestin SHBG ligand, or one of multiple SHBG ligands, is ethinyl estradiol (EE) and wherein the composition is formulated to deliver less than 2.5 micrograms of the estrogen per day.

37. The kit of claim 32, wherein the composition comprises at least one non-progestin SHBG ligand other than EE, wherein the amount of the SHBG ligand included is an amount equivalent to the amount of EE required to achieve the same portion of displacement of the progestin from the SHBG.

38. The kit of claim 32 wherein the composition comprises two or more non-progestin SHBG ligands other than EE, wherein the sum of the amounts of the SHBG ligands included is an amount equivalent to the amount of EE required to achieve the same portion of displacement of the progestin from the SHBG.

39. The kit of claim 32, wherein the progestin is norgestrel, levonorgestrel, norethindrone, norethindrone acetate or norethrynodrel.

40. The kit of claim 32 wherein the SHBG ligand is not an estrogen and is an estrogenic compound, a non-estrogenic hormone, an anti-SHBG antibody or fragment thereof, a small molecule, or a combination thereof.

41. The kit of claim 32, wherein the SHBG ligand is a combination of an estrogen with one or more of an estrogenic compound, a non-estrogenic hormone, an anti-SHBG antibody or fragment or a small molecule.

42. The kit of claim 32, wherein the SHBG ligand is EE or 17-beta estradiol, in combination with a non-estrogen SHBG ligand.

43. A method of contraception, comprising:
(a) administering to a woman on a regular or continuous basis, during a treatment cycle of duration selected by the woman, a progestin with binding affinity to sex hormone binding globulin (SHBG); and
(b) in a time proximity of between about 12 hours before and about 6 hours after the woman engages in sexual intercourse, administering to the woman a bolus of one or more non-progestin SHBG ligands that bind to SHBG in an amount sufficient to displace at least a portion of the progestin bound to SHBG, thereby increasing the amount of unbound progestin circulating in the blood plasma of the woman, thereby increasing the contraceptive efficacy of the progestin being administered on the regular or continuous basis during the time frame in which the woman could become pregnant due to engaging in sexual intercourse.

44. The method of claim 43, wherein the treatment cycle comprises a treatment interval of between three and twelve weeks, followed by a one-week rest interval in which no hormone is administered, or in which low dose hormone is administered.

45. The method of claim 43, wherein the progestin is norgestrel, levonorgestrel, norethindrone or norethrynodrel.

46. The method of claim 43, wherein the bolus of the SHBG ligand delivered to the woman comprises the equivalent of about 20-100 micrograms of EE.

47. The method of claim 43, wherein the progestin is formulated in a composition for administration by a route selected from oral, transmucosal, transdermal and subcutaneous.

48. The method of claim 47, wherein the progestin composition is formulated in a transdermal delivery device comprising an active ingredient (AI) layer containing the progestin, wherein the AI layer has a skin-contacting surface and a non-skin-contacting surface, and the device further comprises a backing layer adjacent the non-skin-contacting surface.

49. The method of claim 43, wherein the progestin is formulated in a composition that further comprises a non-progestin SHBG ligand in an amount that delivers an equivalent of less than 10 micrograms per day of EE.

50. The method of claim 43, wherein the bolus of SHBG ligand is formulated for oral delivery.

51. A kit comprising:
(a) a multiplicity of dosage units of progestin having SHBG binding affinity formulated in a composition for regular or continuous administration via oral, transmucosal or transdermal delivery;
(b) a multiplicity of dosage units of non-progestin SHBG ligand formulated in a composition for administration as a bolus via oral delivery; and
(c) instructions for use of the kit components in method of contraception comprising: (i) administering to a woman on a regular or continuous basis, during a treatment cycle of duration selected by the woman, a progestin with binding affinity to sex hormone binding globulin (SHBG); and (ii) in a time proximity of between about 12 hours before and about 6 hours after the woman engages in sexual intercourse, administering to the woman a bolus of one or more non-progestin SHBG ligands that bind to SHBG in an amount sufficient to displace at least a portion of the progestin bound to SHBG, thereby increasing the amount of unbound progestin circulating in the blood plasma of the woman, thereby increasing the contraceptive efficacy of the progestin being administered on the regular or continuous basis during the time frame in which the woman could become pregnant due to engaging in sexual intercourse.

52. The kit of claim 51, wherein the progestin is norgestrel, levonorgestrel, norethindrone, norethindrone acetate, or norethrynodrel.

53. The kit of claim 51, wherein the bolus of the SHBG ligand delivered to the woman comprises the equivalent of about 20-100 micrograms of EE.

54. The kit of claim 51, wherein the progestin composition is formulated in a transdermal delivery device comprising an active ingredient (AI) layer containing the progestin, wherein the AI layer has a skin-contacting surface and a non-skin-contacting surface, and the device further comprises a backing layer adjacent the non-skin-contacting surface.

55. The kit of claim 51, wherein the progestin is formulated in a composition that further comprises another SHBG ligand in an amount that delivers an equivalent of less than 10 micrograms per day of EE.

56. A method of increasing the amount of circulating progestin in the serum of a patient administered a progestin, comprising: (a) administering to the patient a progestin having binding affinity to sex hormone binding globulin (SHBG), whereby upon delivery of the progestin to the serum of the patient, at least a portion of the progestin is bound to the SHBG and thereby sequestered from circulation in the patient's serum; and (b) co-administering to the patient one or more non-progestin SHBG ligands in an amount sufficient to displace at least part of the progestin from SHBG in the patient's serum, thereby increasing the amount of circulating progestin in the serum of the patient.

57. A method of increasing the potency of a progestin that binds to SHBG, said method comprising co-administering the progestin with a subclinical amount of a non-progestin SHBG ligand other than a progestin.

58. A method of increasing the contraceptive efficacy of a progestin that binds to SHBG, said method comprising co-administering the progestin with a subclinical amount of a non-progestin SHBG ligand.

59. The method of any one of claims 56, 57 and 58, wherein the non-progestin SHBG ligand is an estrogen and is administered in an amount that results in delivery of less than 10 micrograms per day of the estrogen.

60. The method of claim 59, wherein the estrogen is EE and is administered in an amount that results in delivery of less than 2.5 micrograms per day of the EE.
